(19) United States
(12) Patent Application Publication
(10) Pub. No.: US 2013/0217995 A1
(43) Pub. Date: Aug. 22, 2013

(54) Photoacoustic Tomography of Breast Tissue Using Hemispherical Array and Planar Scanning

(71) Applicant: Optosonics, Inc., (US)

(72) Inventor: Robert A. Kruger, Oriental, NC (US)

(73) Assignee: Optosonics, Inc., Oriental, NC (US)

(21) Appl. No.: 13/692,387

(22) Filed: Dec. 3, 2012

Related U.S. Application Data
(60) Provisional application No. 61/565,870, filed on Dec. 1, 2011.

(51) Int. Cl. A61B 5/00

(52) U.S. Cl. CPC A61B 5/0095; A61B 5/4312
USPC 600/407

(57) ABSTRACT

Photoacoustic imaging is enhanced by scanning (61) the sensor array (10) used in photoacoustic imaging laterally relative to the tissue being imaged, gathering multiple tissue images (70, 71, 72, 73) at multiple relative lateral positions, and generating a photoacoustic image (80) of the tissue by combining the images taken at multiple relative lateral positions.

### FIELD OF THE INVENTION

The present invention relates to photoacoustic computed tomography (PAT) which is also known as photoacoustic computed tomography (PCT) or optoacoustic tomography (OAT).

### BACKGROUND OF THE INVENTION

The absorption of light from a modulated light source can be used to stimulate acoustic emissions in biologic tissue, e.g., breast tissue, wherever the light is absorbed. One common light source is a laser operating in the near-infrared region of the electromagnetic spectrum (wavelength=700-1200 nm). The most common type of optical modulation employs short-duration pulses (5-200 nanoseconds), pulsed at a rate of 10-1000 times per second. The 25 subsequent acoustic emissions typically lie in the medical ultrasound frequency range (1,000,000-20,000,000 cycles per second). These emissions propagate throughout the tissue at approximately 1500 meters per second and may subsequently be detected by an array of acoustic sensors placed outside the tissue surface. Typically, these arrays consist of 64-512 sensors mechanically affixed to a curved surface, usually spherical or cylindrical, but other curved surfaces may be used as well. It is possible to form three-dimensional (3D) images of the pattern of optical absorption in the tissue by using mathematical algorithms for image reconstruction applied to the detected acoustic waves, techniques commonly referred to as photoacoustic tomography (PAT).

Descriptions of such techniques can be found in U.S. Pat. Nos. 5,713,356 and 6,102,857, which are hereby incorporated by reference herein in their entirety. Significantly, 3D-PAT images formed in this way primarily depict blood vessels and tumors, because light in the near infrared is predominantly absorbed by hemoglobin, which is concentrated in blood and malignant tumors. They can also be used to detect optically absorbing contrast agents that are administered intravenously.

The Applicant herein has previously obtained patents relating to the use of ultrasound and photoacoustic ultrasound in the imaging of tissue, including U.S. Pat. Nos. 5,713,356; 6,102,857; 6,104,942; 48 6,216,025; 6,292,682; 6,490,470; 6,633,774 and 7,774,042. All of these prior patents are hereby incorporated herein by reference.

### SUMMARY OF THE INVENTION

Previous embodiments of three dimensional PAT imaging using a hemispherical array employed a stationary beam of light that illuminates part of the tissue being imaged, e.g., a breast. As the hemispherical array is rotated about a vertical axis, the light source is pulsed. In this geometry, each element of the detector array “points” toward the center of curvature of the hemisphere. This common point lies at the intersection of rays passing through the centers of each flat, disk-shaped transducer, whose surfaces are oriented 90 degrees to the rays intersecting their centers. These detectors have the property that they are most sensitive to photoacoustic signals that impinge their front surface from the direction of the center of curvature, the “on-axis” direction. The transducer exhibits decreasing sensitivity off-axis as the off-axis angle increases. Consequently, the PAT imaging system detects photoacoustic signals from tissue located close to the center of curvature of the array with the greatest sensitivity, and that sensitivity decreases as the distance from the center of rotation increases. Thus, the PAT system produces a useful 3D image only within a limited volume, centered at the center of rotation.

The volume that can be imaged by a PAT system of prior embodiments can be increased by increasing the radius of the hemispherical array, but to image a large volume of tissue, e.g., a 1000 mL breast, the size of the hemisphere would become prohibitively large. One can also decrease the physical size of the transducers (typically 3-5 mm diameter), thereby increasing their off-axis sensitivity, but this results in an undesirable reduction in overall sensitivity, potentially below what is needed to detect the typically weak photoacoustic signals produced within tissue.

In accordance with principles of the present invention, an alternative strategy is applied to the sensitivity challenges in the prior embodiments of a PAT scanner. Specifically, the sensitivity is improved by scanning the hemispherical array laterally within a plane, e.g., in a rectilinear fashion (left-right, back-forth) as the photoacoustic data are acquired. Importantly, this planar scanning is implemented independently from the rotational scanning of the hemispherical array known in the prior embodiments. In some embodiments, the rotation and scanning occur together, and in other embodiments, the hemispherical array may not be rotated at all during the planar scan. In either case, the net effect is to position the sensitive volume of the scanner variously throughout a larger volume of tissue than can be imaged with the hemispherical array in a fixed location and thus always pointing at the same volume throughout a scan

Planar scanning in accordance with principles of the present invention can be accomplished either by moving the array beneath a stationary exam table, supporting the patient being imaged, or by moving the exam table supporting the table above a stationary array, which may be allowed only to rotate.

The above-described and other advantages will be apparent in light of the following figures and detailed description of principles of the present invention.

### BRIEF DESCRIPTION OF THE DRAWINGS

The accompanying drawings, which are incorporated in and constitute a part of this specification, illustrate embodiments of the invention and, together with a general description of the invention given above and the detailed description of the embodiments given below, serve to explain the principles of the invention.

FIG. 1 is a cross-sectional diagram showing the overall structure of a PAT scanner;

FIG. 2 illustrates details of the geometry of the hemispherical sensor array of the scanner of FIG. 1;

FIG. 3 is a graphical depiction of the angular sensitivity of the hemispherical array of FIG. 2;

FIG. 4 is an illustration of a target used for uniformity testing of a PAT scanner;

FIG. 5 is a slice of an image of the target of FIG. 4 taken with the PAT scanner of FIG. 1 centered over the target;

FIG. 6 is a schematic diagram showing the lateral displacement that may be applied to the hemispherical sensor array of FIG. 2 to expand the field of view in accordance with principles of the present invention;

FIGS. 7A, 7B, 7C and 7D illustrate four independent PAT images taken at each laterally displaced position shown in FIG. 6; and

FIG. 8 is a composite image of the target of FIG. 4 formed by the sensor array at the four laterally displaced positions shown in FIG. 6, showing greater detail of the target than in FIG. 5.

It should be understood that the appended drawings are not necessarily to scale, presenting a somewhat simplified representation of various preferred features illustrative of the basic principles of the invention. The specific design features of the sequence of operations as disclosed herein, including, for example, specific dimensions, orientations, locations, functions and shapes of various illustrated components, will be determined in part by the particular intended application and use environment. Certain features of the illustrated embodiments may have been enlarged or distorted relative to others to facilitate visualization and clear understanding.

### DETAILED DESCRIPTION

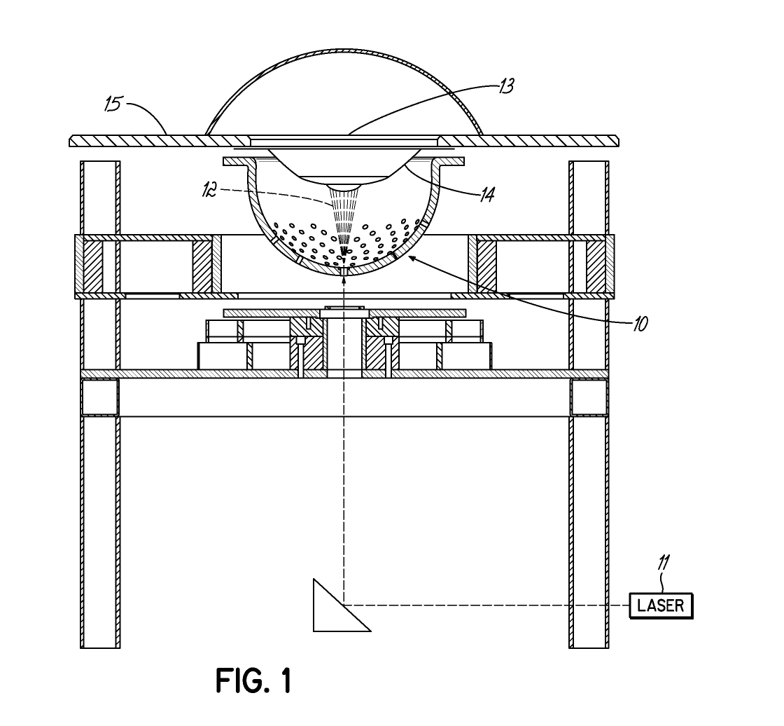

FIG. 1 illustrates the basic elements that comprise a PAT scanner. A liquid-filled, hemispherical, detector array 10 detects photoacoustic signals that are emitted from tissue in response to a pulsed laser 11 that produces a light beam 12 that illuminates the tissue 13 being imaged. The tissue is restrained by an acoustically and optically transparent, plastic membrane 14 affixed to a tabletop 15 upon which the patient lies. The laser 11 is pulsed at a typical rate of 10 times per second (10 Hertz) as the detector array 10 is rotated about the vertical axis, completing a full rotation in 3-24 seconds.

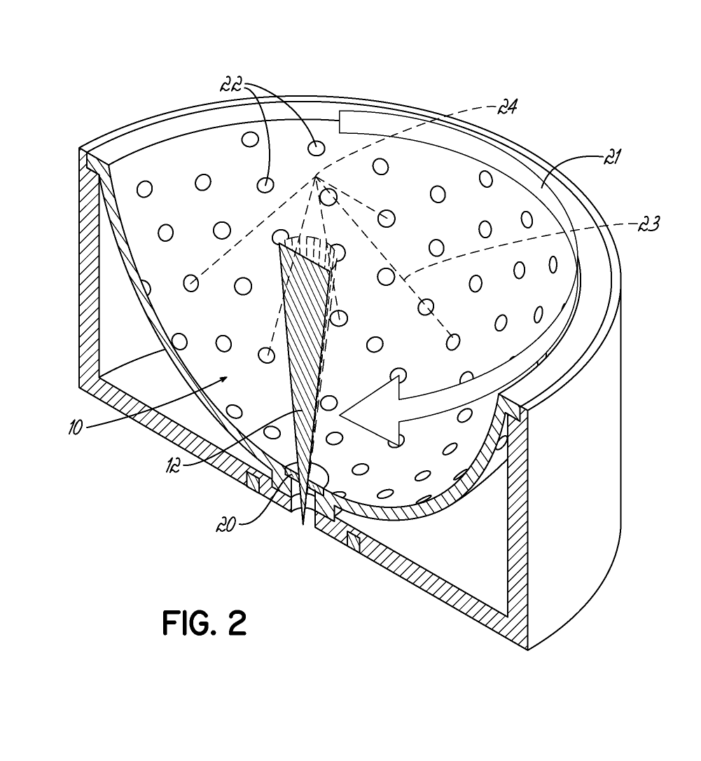

FIG. 2 illustrates details of the hemispherical detector array 10. An optically clear aperture 20 at the base of the hemisphere allows the light beam 12 to illuminate the tissue placed above the array. This hemispherical bowl rotates about the light beam as shown at 21 during data acquisition. Photoacoustic signals are detected by each transducer 22 that comprises the array following each pulse of light. These transducers are flat-faced, and “point” to the center of curvature 24 of the array, where on-axis rays 23 from all the transducers converge.

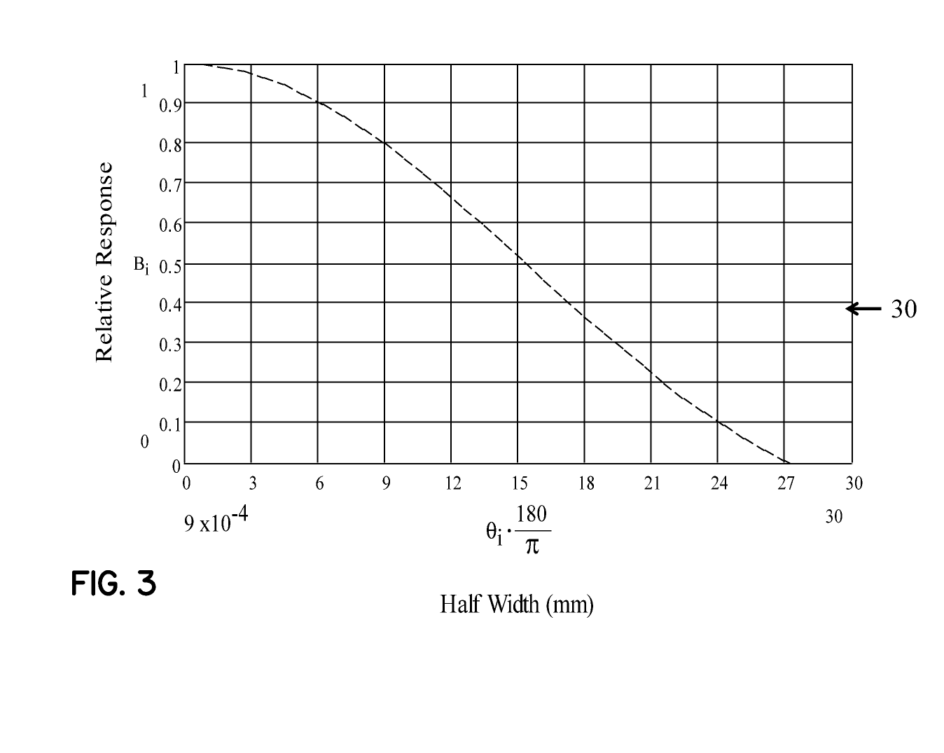

The graph 30 in FIG. 3 of Far-Field Angular Response describes the angular sensitivity, relative to an on-axis ray (23, FIG. 2) for a typical transducer element of which the hemispherical array is comprised. In this case, the transducer is a 2-mm diameter disk with peak acoustic sensitivity at 2 MHz (2,000,000 cycles per second). As is illustrated, this particular transducer has a sensitivity of at least 50% of its peak sensitivity over an angular range of ±15 degrees from perpendicular to the disk. Photoacoustic signals detected within this range of angles of the perpendicular axis of the disk are the most useful for three dimensional PAT imaging.

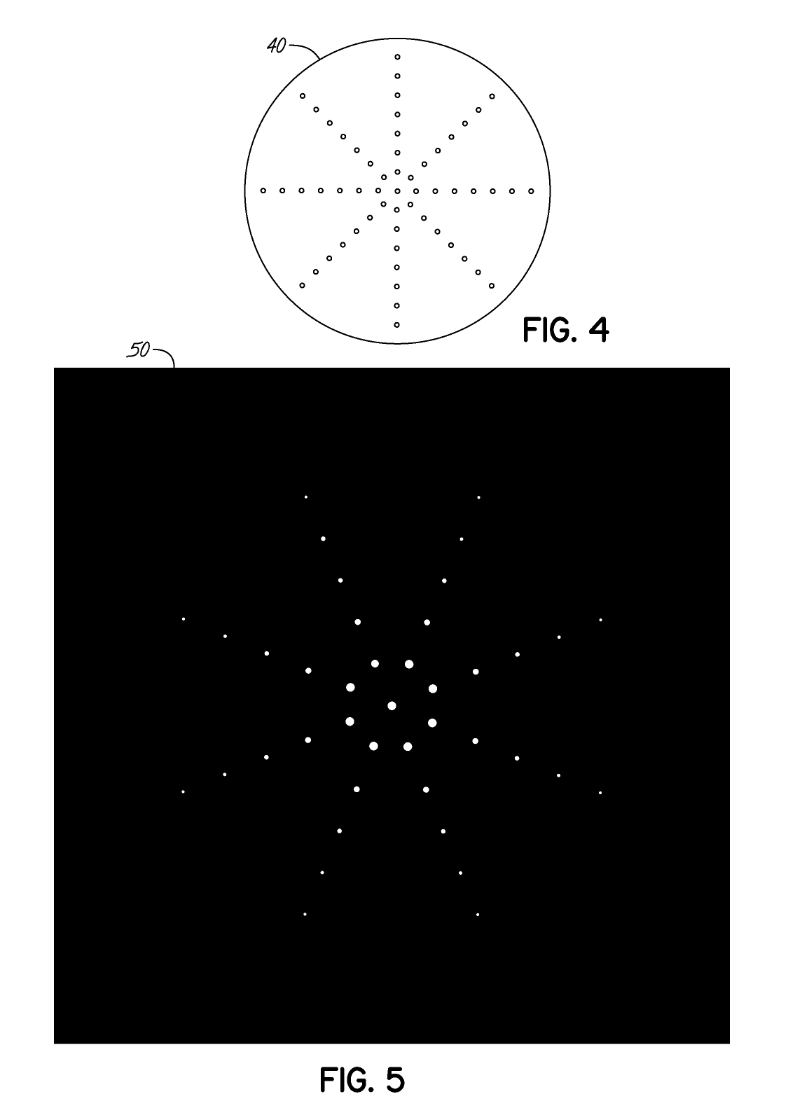

The effective field of view of a three dimensional PAT scanner can be assessed by placing within the scanner, a uniformity target 40, such as the one illustrated in FIG. 4. This target consists of a sheet of clear plastic upon which a pattern of black dots, spaced 5-mm apart radially, have been printed. This target is placed within the liquid-filled, plastic membrane (14, FIG. 2) and a three dimensional PAT image is acquired and reconstructed for viewing in accordance with the imaging methods disclosed in the above referenced patents which are incorporated herein.

One slice (50) from a three dimensional PAT image of the uniformity target (40) is shown in FIG. 5. As is apparent, the field of view is only about +/−20 mm wide (four dots from the center point of the target).

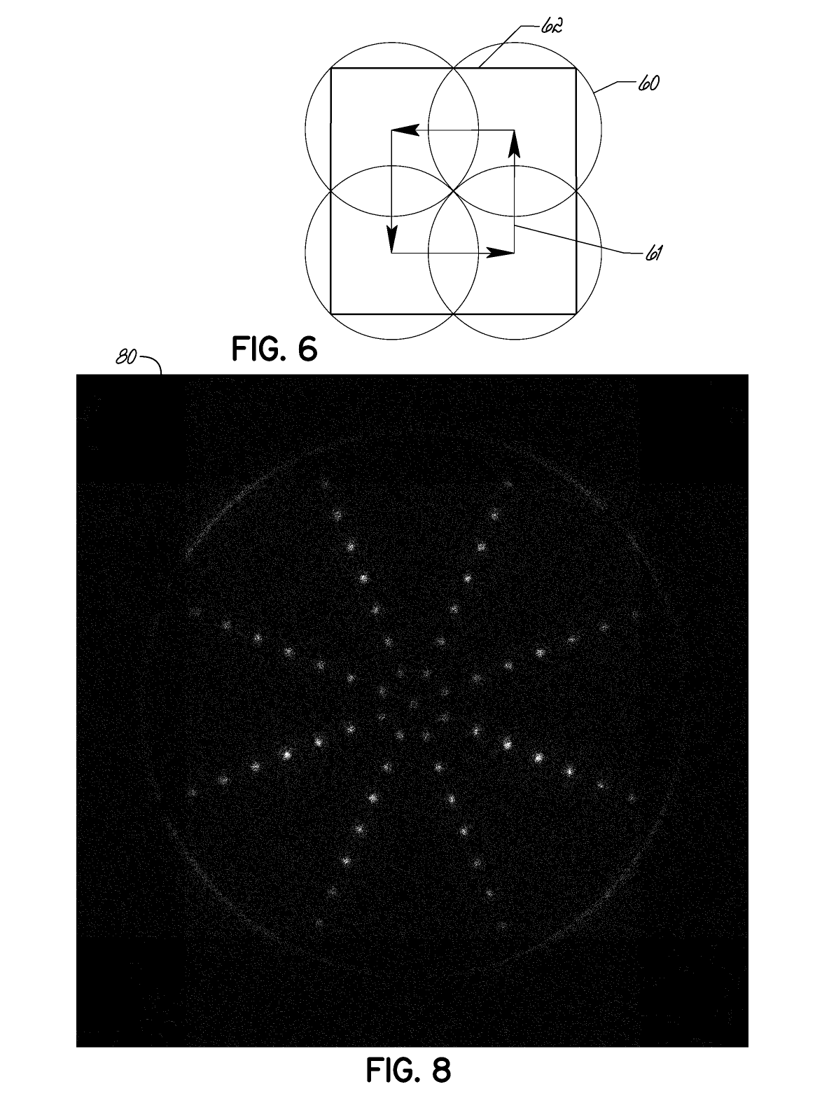

Alternative data acquisition in accordance with principles of the present invention uses four sets of PAT data of the uniformity phantom 40, where the light field 60 has been displaced laterally in a 2×2 rectilinear fashion 61 between the four scans, in accordance with the pattern illustrated in FIG. 6, resulting in a composite field of view 62 of greater extent than accomplished without rectilinear scanning.

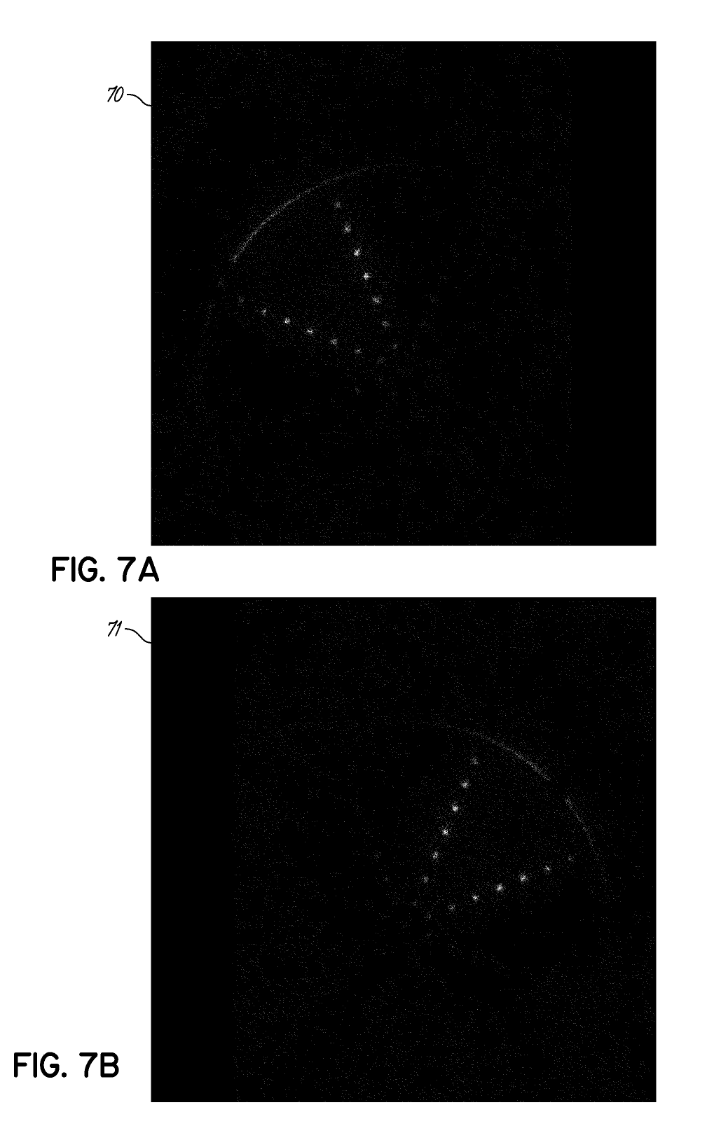

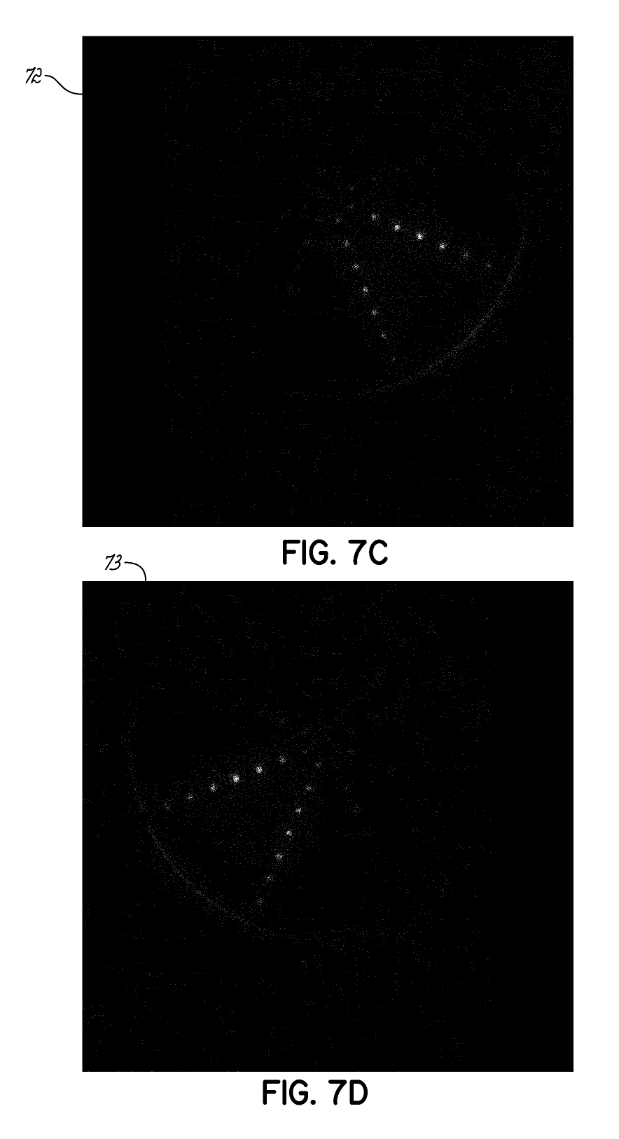

Four independent PAT images 70 (as shown in FIG. 7A), 71 (as shown in FIG. 7B), 72 (as shown in FIG. 7C) and 73 (as shown in FIG. 7D), one for each position of the light beam, are shown in FIG. 6. These were generated by displacing the light field to four locations are arranged in a square pattern 32 mm on a side.

FIG. 8 demonstrates how the field of view of the uniformity phantom (40, FIG. 4) has been increased by the use of rectilinear scanning of the light beam 12 coupled to the hemispherical array 10. Specifically, a composite image 80 is assembled from the four component images (70, 71, 72 and 73, FIG. 7) by shifting each of the component images to compensate for the rectilinear shift 61 from the center of the uniformity phantom 40 used during data acquisition, and then summing the resulting image data together. The field of view seen in FIG. 8 is clearly superior and more uniform in contrast than accomplished without rectilinear scanning.

While embodiments of the present invention have been illustrated by a description of the various embodiments and the examples, and while these embodiments have been described in considerable detail, it is not the intention of the applicants to restrict or in any way limit the scope of the appended claims to such detail. Additional advantages and modifications will readily appear to those skilled in the art. For example, although the array surface has been described as “hemispherical,” other curved or piecewise linear surfaces could also be used. Moreover, rotation and movement of the curved surface to multiple locations may be used to gather data from more virtual transducer locations than there are physical transducers in the apparatus, as elaborated in the above-referenced patents. Accordingly, departures may be made from such details without departing from the spirit or scope of applicants' general inventive concept.

1. A method of photoacoustic imaging of tissue, comprising:
photoacoustically stimulating said tissue with a source of light;
capturing photoacoustically generated acoustic signals from said tissue via a plurality of spaced transducers arranged about the tissue;
repositioning each of the spaced transducers relative to the tissue by a common displacement distance and direction and repeating the steps of stimulating the tissue, capturing photoacoustically generated acoustic signals;
generating an image of the tissue by combining the captured acoustic signals from the capturing steps.

2. The method of claim 1 wherein the transducers are mechanically affixed to a surface.

3. The method of claim 2 wherein the surface is a smoothly curved surface.

4. The method of claim 3 wherein the surface is hemispherical.

5. The method of claim 1 wherein the source of light produces pulsed light.

6. The method of claim 1 wherein the source of light produces modulated pulses of continuously modulating light.

7. The method of claim 1 wherein the light is laser light.

8. The method of claim 1 wherein the source of light comprises a central light source.

9. The method of claim 1 further comprising repeating the steps of repositioning each of the spaced transducers, photoacoustically stimulating the tissue, and capturing photoacoustically generated acoustic signals one or more additional times.

10. The method of claim 1 wherein the step of repositioning each of the spaced transducers relative to the tissue comprises moving the tissue while the sensors remain stationary.

11. The method of claim 1 wherein the step of repositioning each of the spaced transducers relative to the tissue comprises moving the sensors while the tissue remains stationary.

12. The method of claim 1 wherein the step of repositioning each of the spaced transducers relative to the tissue comprises moving the sensors and tissue.

13. The method of claim 1 in which the photoacoustically stimulating step and capturing step are performed before and after but not during the repositioning step.

14. The method of claim 1, wherein the step of generating an image of the tissue comprises generating plural images of the tissue, one image generated from each repetition of the respective capturing and photacoustically stimulating steps, and combining the plural images into a single image of the tissue.

15. The method of claim 14 wherein the step of combining the plural images comprises combining points in each respective image that are spaced by the common displacement distance and direction.

16. The method of claim 1 in which the photoacoustically stimulating step and capturing step are performed during the repositioning step.

17. The method of claim 16 wherein the step of generating an image of the tissue generates a point in the image from captured acoustic signals from plural capturing steps.

18. The method of claim 17 wherein the step of generating an image comprises combining acoustic signals using computed displaced positions of each sensor relative to the tissue at the time that each sensor captured the acoustic signal.

19. The method of claim 1 wherein the repositioning and repeating steps create a rectangular pattern of relative motion between the transducers and tissue.

20. The method of claim 1 wherein the repositioning and repeating steps create a rectangular pattern of relative motion between the transducers and tissue.

21. The method of claim 1 wherein the repositioning and repeating steps create a circular pattern of relative motion between the transducers and tissue.

22. The method of claim 1 wherein the repositioning and repeating steps create a spiral pattern of relative motion between the transducers and tissue.

23. The method of claim 1 further comprising a step of rotationally repositioning each of the spaced transducers relative to the tissue between steps of photoacoustically stimulating and capturing acoustic signals from said tissue.
